(19) United States
(12) Patent Application Publication
(10) Pub. No.: US 2009/0028810 A1
(43) Pub. Date: Jan. 29, 2009

(54) STABILIZATION OF BIOSOLIDS USING IRON NANOPARTICLES

(75) Inventor: Wei-Xian Zhang, Bethlehem, PA (US)

Correspondence Address: RATNERPRESTIA, P.O. BOX 980, VALLEY FORGE, PA, 19482, US

(73) Assignee: LEHIGH UNIVERSITY, Bethlehem, PA (US)

(21) Appl. No.: 11/914,983

(22) PCT Filed: May 25, 2006
(86) PCT No.: PCT/US06/20429

§ 371 Date: Jun. 26, 2008

Related U.S. Application Data
(60) Provisional application No. 60/685,348, filed on May 27, 2005.

(51) Int. Cl. A61L 11/00; A01N 59/16

(52) U.S. Cl. 424/76.6; 424/646

(57) ABSTRACT

This invention discloses a stabilized biosolids composition and a method for the stabilization of biosolids. It entails the use of a chemically and biologically reactive material, in the form of ultrafine iron particles. The nanometer-sized iron particles are capable of degrading odorous organosulfur compounds, transforming persistent and toxic organic pollutants such as PCBs and chlorinated pesticides, inhibiting the growth of pathogens by increasing pH and maintaining the increased pH of the stabilized biosolids, immobilizing toxic metal ions such as mercury and lead, and improving the overall quality of biosolids for land application and plant growth.

### FIELD OF THE INVENTION

The present invention relates to the stabilization and processing of biosolids. In particular, this invention is directed to stabilization of biosolids compositions using iron nanoparticles.

### BACKGROUND OF THE INVENTION

The term “biosolids” refers to organic sludge generated in municipal wastewater treatment. Of the many approaches utilized, land application is the most commonly used disposal or remediation method. However, nuisance odors and the potential of pathogen and toxic chemical transmission have severely limited the practice of land application. Biosolids include, but are not limited to, substantial amounts of organic substances. For example, 59% to 88% by weight of biosolids from activated sludge treatment of municipal wastewater are biological materials and an assortment of organic compounds.

Biosolids are often disposed of, dispersed or distributed on agricultural land, in forests, rangelands, or on disturbed or environmentally impacted land in need of reclamation or remediation. These applications are referred to as land applications. Disposal, dispersal or distribution of biosolids through land application serves many beneficial purposes. It provides an economic method for the disposal of large volumes of biosolids generated everyday. Land application minimizes soil erosion, improves soil properties, including but not limited to texture and water holding capacity, making conditions on the treated land more favorable for root growth and increasing drought tolerance of vegetation. Biosolids also include nutrients and elements essential for plant growth, including but not limited to nitrogen and phosphorous (Table 1), as well as other essential nutrients including but not limited to iron and zinc. The nutrients in biosolids compositions serve as alternatives to, or substitutes for, expensive chemical fertilizers. Furthermore, the nutrients in biosolids offer certain advantages over those nutrients available in inorganic fertilizers because they are biological and organic in character and are released slowly to growing plants. Biological and organic forms of nutrients are less water soluble and, therefore, less likely to leach into groundwater or runoff into surface water. EPA estimates that more than 7 million dry tons of biosolids are generated annually for use, disposal, dispersal or distribution by the over 16,000 wastewater treatment facilities in the U.S., of which approximately 60% are land applied, composted, or used as landfill cover.

TABLE 1
Comparison of nutrient levels in commercial fertilizers and biosolids

| | Nitrogen | Phosphorus | Potassium |
|---|---|---|---|
| Fertilizers for | 5% | 10% | 10% |
| typical agricultural | | | |
| use | | | |
| Typical biosolids | 1.6-3.0% | 1.5-4.0% | 0-3% |

International Patent Publication WO 2003014031 A1 discloses a method of disinfecting and stabilizing organic wastes where organic waste is intimately mixed with one or more mineral by-products to produce a mixture having a pH of less than about 9. The mixture is heated and dried to produce a stable, granular bio-mineral product that may be used for example, as a fertilizer, soil amendment or as a soil substitute.

### SUMMARY OF THE INVENTION

The present invention provides stabilized biosolids compositions by combining biosolids with nanometer sized metal particles having high surface areas. A surface promoted reaction of biological materials and organic compounds with the ultrafine metal particles (ultrafine defined herein as particles having particle sizes <1 μm), including but not limited to iron particles, provides stabilized biosolids having reduced or minimal amounts of odorous and halogen containing biological materials and organic compounds in addition to inhibiting the growth of one or more pathogens. The iron particles are capable of degrading odorous inorganic sulfur and organosulfur compounds, chemically transforming (thus minimizing or eliminating) persistent and toxic organic pollutants such as polychlorinated biphenyls (PCBs) and chlorinated pesticides, immobilizing toxic metal ions such as mercury ions and lead ions, and improving the overall quality of the treated biosolids for land application and plant growth. Iron is an environment-friendly element and its compounds have a low adverse environmental impact. In fact, iron is an essential element for human, animal and plant growth. This invention offers significant advantages over prior art methods in the speed of treatment and processing of biosolids, in the efficiency of controlling and minimizing odors associated with biosolids, in the simplicity of implementing biosolids processing, in reducing overall operational costs associated with the processing of biosolids.

Accordingly, the present invention provides a stabilized biosolids composition comprising oxidizable iron particles having diameters between 1 to 200 nm and having specific surface areas from 1000 to 763,358 m²/kg.

The present invention also provides a method for stabilizing and reducing or eliminating one or more pathogens in a biosolids composition including the step of combining a biosolids composition with iron particles having diameters between 1 to 200 nm to form a stabilized biosolids composition, as compared to an untreated biosolids composition.

The present invention also provides a method for removing or eliminating odors associated with one or more pathogens in a biosolids composition including the step of treating a biosolids composition with iron particles having diameters between 1 to 200 nm, reducing or eliminating odorous biological and organic compounds, as compared to an untreated biosolids composition.

The present invention also provides a method for dehalogenating one or more halogen containing compounds comprising the steps of combining the biosolids composition with iron particles having diameters between 1 to 200 nm, and lowering or eliminating the halogen content, as compared to an untreated biosolids composition.

The present invention also provides a method for increasing, controlling or maintaining pH of a biosolids composition including the step of combining the biosolids composition with iron particles having diameters between 1 to 200 nm, as compared to an untreated biosolids composition.

### BRIEF DESCRIPTION OF THE DRAWINGS

FIG. 1 summarizes how iron nanoparticles serve as a versatile reagent for the treatment of biosolids.

FIG. 2 is a transmission electron micrograph of a single iron nanoparticle (˜66 nm in diameter).

FIG. 3 is a transmission electron micrograph of iron nanoparticles synthesized by the reduction of ferric iron (Fe³⁺) using sodium borohydride

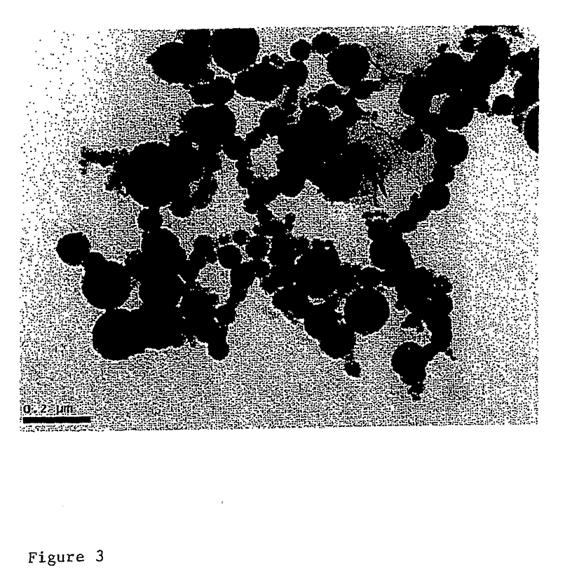

FIG. 4 summarizes changes of solution pH as a function of time and concentration of iron nanoparticles.

FIG. 5 summarizes changes of solution Eₕ as a function of time and concentration of iron microparticles.

FIG. 6 summarizes a Eₕ-pH diagram of Fe(0) in water.

FIG. 7 summarizes immobilization of metal ions (“Mⁿ⁺) with nanoscale iron particles.

FIG. 8 summarizes structures of the environmentally significant HCH isomers, including the two HCH enantiomers.

FIG. 9 summarizes the effectiveness of nanoscale iron (“nFe”) particles in removing the HCHs from solution as a function of dosage.

FIG. 10 is a schematic diagram of an iron particle feeding and mixing system in combination with conventional lime stabilization.

### DETAILED DESCRIPTION OF THE INVENTION

Biosolids refer to sludge produced in various wastewater treatment processes. Biosolids consist primarily of organic substances. For example, 59%-88% by weight of biosolids from activated sludge treatment of municipal wastewater are organic materials.

Biosolids are often disposed on agricultural land, forests, rangelands, or disturbed land in need of reclamation. This is known as land application. Disposal of biosolids through land application serves many beneficial purposes. It provides an economic method for the disposal of large volumes of biosolids generated everyday. Land applications reduce the erosion of soil, improve soil properties, such as texture, and water holding capacity. This makes conditions more favorable for root growth and increases the drought tolerance of vegetation. Biosolids also contain nutrients essential for plant growth, including nitrogen and phosphorous (Table 1), as well as other essential nutrients such as iron and zinc. The nutrients in the biosolids serve as alternatives or substitutes for expensive chemical fertilizers. Furthermore, the nutrients in the biosolids offer certain advantages over those in inorganic fertilizers because they are organic and are released slowly to growing plants. Organic forms of nutrients are less water soluble and, therefore, less likely to leach into groundwater or runoff into surface water.

EPA estimates that more than 7 million dry tons of biosolids are generated annually for use or disposal by the 16,000 wastewater treatment facilities nationwide. Of this, approximately 60% are land applied, composted, or used as landfill cover.

Federal regulations require that biosolids be processed before they are applied to land. The treatment processes are often termed as “stabilization” as they help minimize odor generation, destroy pathogens (disease causing organisms), and reduce vector attraction potential. Details of the federal regulations can be found in “Standard for the Use and Disposal of Sewage Sludge, the Part 503 Rule” by U.S. Environmental Protection Agency.

Nuisance odor is often the number one complaint/objection associated with land applications. It is encountered frequently when the beneficial use sites are close to residential areas. Odors are mostly generated by biological activities in the biosolids with various biodegradable organic substances. For example, biosolids from activated sludge treatment contains a variety of proteins. Naturally, biosolids are a rich source of food for microorganisms. Due to the lack of oxygen, the biological reactions in biosolids are mostly anaerobic producing various odorous compounds. Organic and inorganic compounds of sulfur and nitrogen (Table 2) produce the most offensive odor causing compounds in biosolids.

TABLE 2
Examples of odorous compounds commonly found in biosolids

| Compound | Formula | Threshold (ppm) |
|---|---|---|
| Ammonia | NH₃ | 46.8 |
| Hydrogen sulfide | H₂S | 0.00047 |
| Meth I merca ton | CH₃SH | 0.0021 |
| Dimeth I sulfide | CH₃CH₃ | 0.0001 |
| Indole | C₈H₆NH | 0.0001 |

Biosolids also contain various metals in the form of water soluble metal cations. Those metals can be detrimental to plants and animals. The term “heavy metal” has been often used to denote several of trace metals in biosolids. Concentrations of heavy metals vary widely as indicated in Table 3. For land applications, the presence of heavy metals limits both the application rate and useful life span of the disposal site.

Biosolids also have a large variety of microorganisms and viruses. Some are disease-causing pathogens such as bacteria (salmonella sp., vibrio cholerae), protozoa (Giardia lamblia), viruses (hepatitis and Norwalk). Table 3 gives examples of common pathogens in biosolids.

TABLE 3
Examples of pathogens potentially present in biosolids.

| Organism | Disease | Symptoms |
|---|---|---|
| Bacteria | | |
| E. Coli | Gastroenteritis | Diarrhea |
| Legionella pneumophila | Legionnaires' | Malaise, fever, |
| | disease | myalgia, etc. |
| Salmonella | Salmonellosis | Food poisoning |
| Protozoa | | |
| Cryptosporidium parvum | Cryptosporidiosis | Diarrhea |
| Giardia lamblia | Giardiasis | Diarrhea, nausea |
| Balantidium coli | Balantidiasis | Diarrhea sent |
| Helminthes | | |
| Ascaris lumbricoides | Ascariasis | Roundworm infestation |
| Enterobius vermicularis | Enterobiasis | Pineworm |
| T. solium | Taeniasis | Park tapeworm |
| Viruses | | |
| Adenovirus | Respiratory disease | |
| Enteroviruses | Gastroenteritis etc. | |
| Hepatitis A virus | Infectious hepatitis | |

Conventional methods used in the treatment of biosolids include biological digestion, alkaline treatment, composting, heat drying and pelletizing (Table 4). These methods are often expensive, time consuming, and ineffective in terms of abating odors and immobilizing heavy metals associated with biosolids.

TABLE 4
Common Methods for Biosolids Stabilization

| Treatment Processes | Use or Disposal Methods |
|---|---|
| Aerobic or Anaerobic | Produces biosolids used as s soil |
| Digestion | amendment and organic fertilizer on |
| | pasture and row crops, forests, and |
| | reclamation sites. |
| Alkaline Treatment | Produce biosolids useful for land |
| | application and for use as daily landfill |
| | cover. |
| Composting | Produces highly organic, soil-like biosolids |
| | with conditioning properties for |
| | horticultural, nursery, and landscape uses. |
| Heat-Drying/Pelletizing | Produces biosolids for fertilizers generally |
| | used at a low rate because of higher cost |
| | and higher nitrogen content |

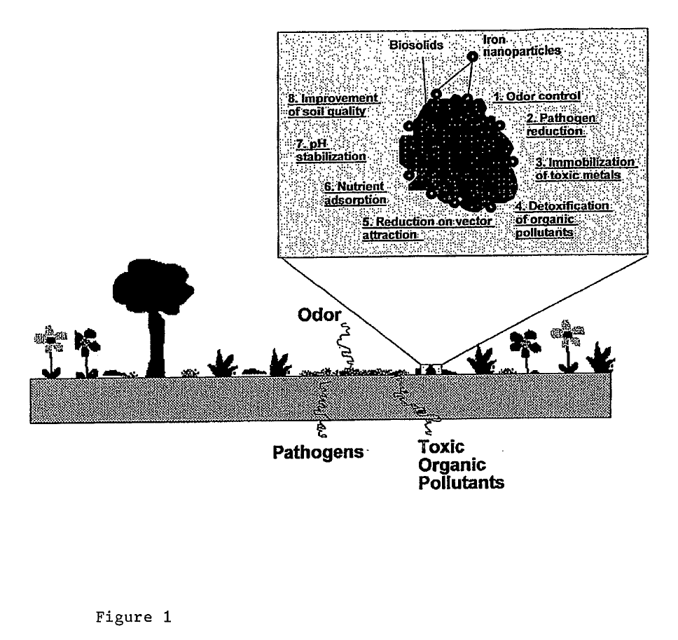

The present invention provides a new procedure for biosolids stabilization. Specifically, reactive metal particles, especially ultra fine, nanometer sized iron nanoparticles are useful for degrading and stabilizing the above-described pollutants (FIG. 1). The oxidizable iron nanoparticles are useful, according to an exemplary embodiment, for treating biosolids.

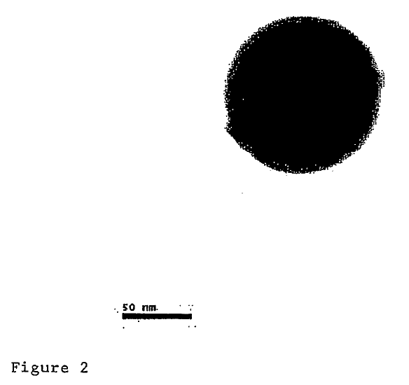

Nanometer sized or nanoscale iron particles in general refer to iron particles having diameters in the range of from 1 to 1000 nm, including from 1 to 500 nm, also including from 1 to 200 nm, including from 1 to 100 nm. The metal nanoparticles are characterized by transmission electron microscopy, for example, to determine particle size (FIG. 2).

According to one exemplary embodiment, the iron particles are combined with other metal particles having diameters from 1 to 200 nm. According to a separate embodiment, the iron particles are combined with other metal particles having diameters from 200 nm to 1,000,000 nm (1,000 μm). For many environmental applications, the reactive component is the metallic or zero-valent iron (Fe^{O}).

Zero-valent iron (Fe^{O}) is a reactive material, which readily reacts with dissolved oxygen:

2Fe^{o}(ₛ)+4H⁺_{(aq)}+0_{2(aq)} [symbol] 2Fe⁺²_{(aq)}+2H₂O (1)

Equation 1 summarizes a classical corrosion reaction by which iron is oxidized from exposure to oxygen and water. In accordance with the invention, iron functions as an efficient and relatively inexpensive electron donor, as shown in equation 2:

Fe [symbol] Fe²⁺+2e⁻ (2)

Oxidized ferrous iron (Fe(II)) can be further oxidized to ferric iron (Fe(III)). Although oxygen and water are common electron acceptors in the environment, other substances including many environmental contaminants can serve as electron acceptors. For example, trichloroethene (TCE), a common contaminant, can receive the electrons from iron and be reduced to ethene as summarized in equation (3):

C₂HCl₃+3H⁺+6e⁻ [symbol] C₂H₄+3Cl⁻ (3)

In accordance with the present invention, electrons released from iron oxidation are utilized for the stabilization of biosolids. Numerous studies on the hydrodechlorination of chlorinated organic contaminants using iron nanoparticles have been reported. Field tests have demonstrated the potential of iron nanoparticles for in-situ remediation. Recent work has expanded the applications to the remediation of polychlorinated biphenyls (PCBs), perchlorate, nitrate, heavy metal ions such as Cr(VI) and Cd(II), and organochlorine pesticides such as DDT and hexachlorocyclohexane.

Nanoparticles provide novel materials with unique reactivity toward targeted contaminants, enhanced mobility in environmental media, and ease of use. The foremost imperative advantage of iron nanoparticles is the large surface area. Iron oxidation is surface mediated. That is, the larger the iron surface, the higher the reaction rate. Stated in another way, the smaller the particle size, the higher the potential reaction rate. For a spherical particle with a diameter of d, surface area per unit of mass, or specific surface area (SSA) can be calculated by the following equation:

$$S S A = \frac{Surface Area}{Mass} = \frac{π {d}^{2}}{ρ \frac{π}{6} {d}^{3}} = \frac{6}{ρ d} \qquad ( 4 )$$

Where ρ is the density (kg/m³) of iron particles.

For the procedure described in this invention, particles both large and small can be used. However, smaller particles offer the advantage of large reactive surface area per unit of iron mass. Smaller amounts of iron are thus needed to achieve biosolid stabilization. This is particularly true for ultrafine particles with diameters less than 100 nanometers. For example, iron particles having particle sizes of 50 nm have SSA of 15,000 m²/kg (Table 3). In comparison, iron powders having a diameter of 1 mm have a theoretical SSA of only 0.77 m²/kg.

TABLE 5
Theoretical specific surface areas (SSA) of spherical iron particles

| Diameter (d) | | SSA (m²/kg) |
|---|---|---|
| 1 | nm | 763,358 |
| 1 | μm | 763 |
| 1 | mm | 0.763 |

*Calculated using equation (4) with [symbol] at 7860 kg/m³ for iron.

Iron nanoparticles for pH adjustment of biosolids

Iron also reacts with water as summarized in equation (5):

Fe^{o}(ₛ)+2H₂0_{(aq)} [symbol] Fe⁺²_{(aq)}+H_{2(g)}+20H⁻ (5)

According to the above reaction, the iron-mediated reactions generate hydroxyl ions (OH⁻) and result in a characteristic increase in pH.

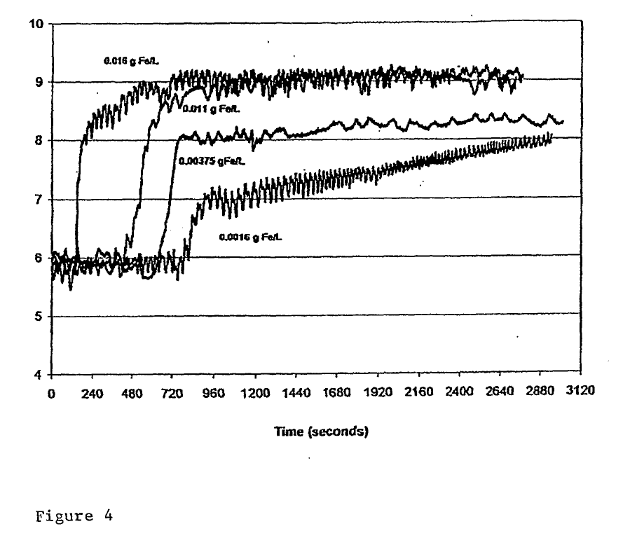

Experimentally measured pH trends in water are illustrated in FIG. 4. FIG. 4 summarizes changes of said solution pH as a function of time and concentration of iron nanoparticles. Typically, the addition of even a small amount (e.g., <1 g/L) of iron particles can raise and maintain the water pH in the range of 8-10.

The pH of the environment is a key factor in the growth of organisms. Most bacteria cannot tolerate pH levels above 9.5 or below 4.0. Generally, the optimum pH for bacterial growth lies between 6.5 and 7.5. Alkaline treatment in which a large amount of lime is added to biosolids has been frequently used in biosolid stabilization. In this context, the addition of iron nanoparticles function to increase pH, inhibiting the growth of pathogens in biosolids.

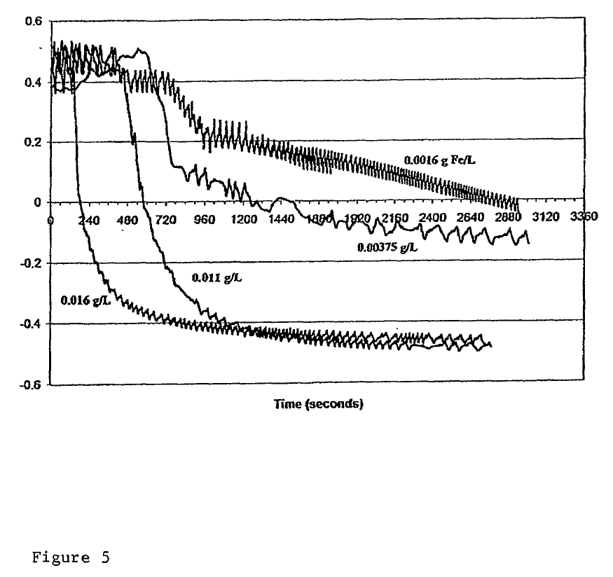

As described above, iron rapidly reacts and consumes oxygen in water. At adequate doses, iron nanoparticles can deplete all dissolved oxygen in biosolids. FIG. 5 summarizes changes of solution oxidation-reduction potentials, Eₕ, as a function of time and concentration of iron microparticles. As shown in FIG. 5, solution oxidation-reduction potential decreases rapidly, indicating a highly reducing environment as a result of the addition of iron nanoparticles. As a result, microorganisms (including pathogens) grow much more slowly in the presence of iron nanoparticles.

Iron Nanoparticles for Controlling Odors in Biosolids

Biosolids often have their own distinctive odor depending on the type of treatment it has been subjected to. Nuisance odors can have detrimental effects on aesthetics, property values and the quality of life in communities subjected to them. Odor complaints often lead to long-term problems. For example, local public opposition can delay or prevent the beneficial reuse of biosolids. Nuisance odors are often the number one complaint associated with land disposal of biosolids.

Compounds that contain sulfur cause most odors. For example, hydrogen sulfide (H₂S) is one compound that contributes to the odor associated with rotten eggs and gives off the characteristic pungent odor. Common odor-causing compounds in biosolids are listed in Table 2. Organic sulfur compounds such as mercaptans and methyl sulfide are identified as the most offensive odor causing compounds associated with biosolids handling and application. These compounds typically are released from biosolids by heat, aeration and digestion.

Sulfur, which is directly below oxygen in the periodic table of the elements, has many properties similar to those of oxygen. One of them is the reaction with metallic iron. For example, sulfate, like oxygen, can accept electrons from iron oxidation and be reduced to elemental sulfur:

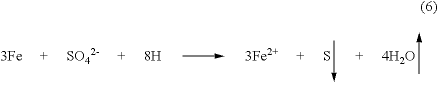

Sulfides (S²⁻) on the other hand precipitate with ferrous iron as both iron sulfide (FeS) and pyrite (FeS₂), and have a relatively low solubility in water:

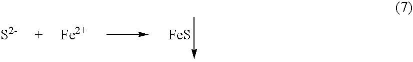

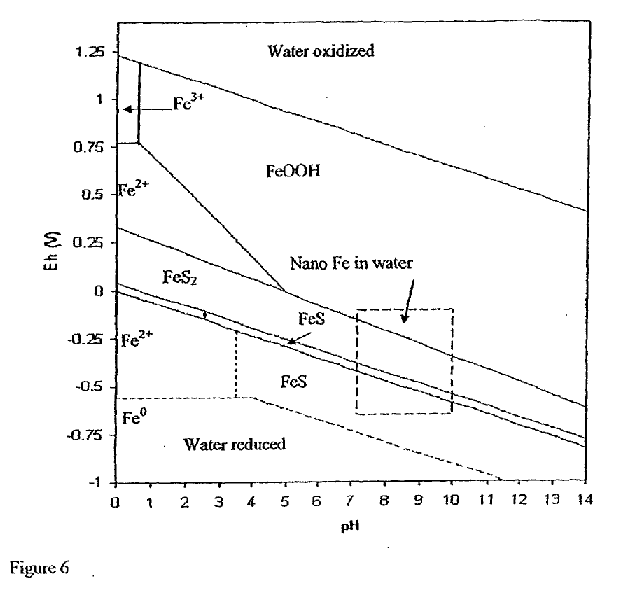

FIG. 6 summarizes oxidation-reduction potentials-pH (Eₕ-pH) diagram of zero valent iron (Fe⁰) in water. Thermodynamically, the most stable forms of sulfur are FeS and FeS₂ (FIG. 6) in the presence of iron nanoparticles.

Iron also reacts with sulfur-containing organic compounds such as dimethyl sulfide, and dimethyl disulfide as summarized in equations (8) and (9):

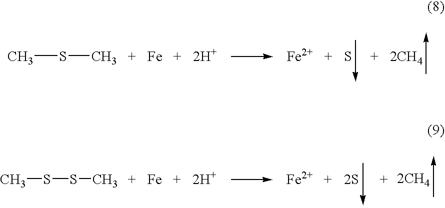

The above reactions point to probable mechanisms for odor reduction where sulfur containing compounds can react and bind to reactive iron particles.

It should be noted that the formation of volatile H₂S is not likely in the presence of zerovalent iron particles. The reactions of iron with water typically increase the solution pH to 8-10 as shown in FIG. 4. Sulfide (S²⁻) likely exists as HS⁻ in this pH range.

Iron for Metal Ion Reduction and Immobilization

As noted above, biosolids contain a large number of metals (Table 6). Among them, federal regulations (Part 503 Rule) have numerical limits on 10 metals. The ten metals are: arsenic, cadmium, chromium, copper, lead, mercury, molybdenum, nickel, selenium, and zinc. As shown in this invention, reactive iron can react and immobilize most regulated metals in biosolids.

TABLE 6
Typical metal contents in biosolids (U.S. EPA 1984)

| | Concentration range | Median concentration |
|---|---|---|
| Metal | (mg/kg) | (mg/kg) |
| Arsenic | 1.1-230 | 10 |
| Cadmium | 1-3,410 | 10 |
| Chromium | 10-99,000 | 500 |
| Cobalt | 11.3-2,490 | 30 |
| Copper | 84-17,000 | 800 |
| Iron | 1,000-154,000 | 17,000 |
| Lead | 13-26,000 | 500 |
| Manganese | 32-9,870 | 260 |
| Mercury | 0.6-56 | 6 |
| Molybdenum | 0.1-214 | 4 |
| Nickel | 2-5,300 | 80 |
| Selenium | 1.7-17.2 | 5 |
| Tin | 2.6-329 | 14 |
| Zinc | 101-49,000 | 1,700 |

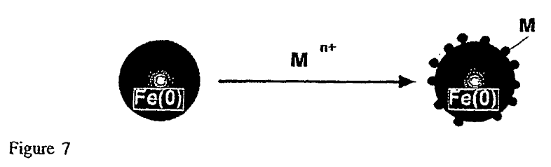

From classical corrosion chemistry, it is known that reactive metals such as iron have relatively low standard potentials and can serve as the electron donors or reductants for the reduction and precipitation of less reactive metal ions. As shown in Table 7, iron is more reactive and can reduce metal ions such as Ni, Pb, Cu, Ag, Cd, and Hg. The reduced metals could precipitate on solid surfaces such as iron and soil particles (FIG. 7). FIG. 7 summarizes immobilization of metal ions Mⁿ⁺ with iron nanoparticles.

TABLE 7
Standard electrode potentials at 25° C. Zero-valent iron can reduce
metal ions with standard potential higher than that of iron (−0.41 V).

| | | EO (volts) |
|---|---|---|
| Zinc (Zn) | Zn²⁺ + 2e− <=> Zn | −0.76 |
| Iron (Fe) | Fe²⁺ + 2e− <=> Fe | −0.41 |
| Cadmium (Cd) | Cd²⁺ + 2e− <=> Cd | −0.40 |
| Cobalt (Co) | Co²⁺ + 2e− <=> Co | −0.28 |
| Nickel (Ni) | Ni²⁺ + 2e− <=> Ni | −0.24 |
| Tin (Sn) | Sn²⁺ + 2e− <=> Sn | −0.13 |
| Lead (Pb) | Pb²⁺ + 2e− <=> Pb | −0.13 |
| Copper (Co) | Cu²⁺ + 2e− <=> Cu | 0.34 |
| Silver (Ag) | Ag⁺ + e− <=> Ag | 0.80 |
| Mercury (Hg) | Hg²⁺ + 2e− <=> Hg | 0.86 |
| Chromium (Cr) | Cr₂0₇²⁻ + I4H⁺ + 6e⁻ <=> 2Cr³⁺ + 7H₂O | 1.36 |

For example, if iron nanoparticles are added to biosolids containing nickel (Ni), Ni(II) can be reduced and precipitated out of water as summarized in equation (10):

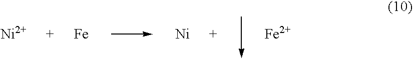

The reduced toxic metals have low solubility in water and thus are less likely to leach into groundwater and runoff into surface water. Bioavailability and biotoxicity of the reduced metals are expected to be lower too. According to one exemplary embodiment of the invention, the oxidizable iron particles reduce and immobilize or eliminate toxicity associated with metal ions selected from the group of metals consisting of: cadmium, copper, lead, nickel, cobalt, mercury, chromium, and combinations of metal ions.

According to one exemplary embodiment, reduction and immobilization of chromium is used as an example to demonstrate the ability of iron nanoparticles for metal ion stabilization.

Chromium is one of the most commonly used metals and also one of the frequently detected inorganic contaminants in soil and water. Chromium has high and acute toxicity to humans, animals, plants, and microorganisms, and is classified as a potential carcinogen.

Chromium in natural waters exists primarily in +3 and +6 valence states. Hexavalent chromium, Cr(VI), such as chromate (2CrO₄²⁻) is highly soluble and mobile in aquatic systems. On the other hand, trivalent chromium [Cr(III)] is relatively stable and has low solubility (<10⁻⁵ M) in aqueous solutions over a wide pH value range. Hexavalent chromium can be reduced to trivalent chromium by iron nanoparticles as shown in equation (11):

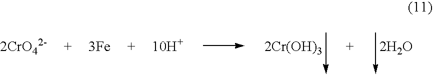

Table 8 presents results of hexavalent chromium reduction by iron nanoparticles from a laboratory experiment with contaminated groundwater and soil samples. Groundwater and soil samples were collected from an industrial site in New Jersey. The groundwater contained 42.83±0.52 mg Cr/L, and the soil had 3,280±90 mg Cr/kg. This study shows that one gram of nanoparticles can reduce 84.4-109.3 mg Cr(VI) in the groundwater and 69.28-72.65 mg Cr(VI) in soil/groundwater slurries, respectively. This reduction capacity is 50-70 times greater than that of microscale under similar experimental conditions.

TABLE 8
Reductive capacity of Cr(VI) by Iron

| | Type of Fe | |
|---|---|---|
| | particles | mgCr(VI)/g Fe |
| Groundwater | Micron-sized Fe | 1.53-1.75 |
| | Nano-sized Fe | 84.40-109.30 |
| Soil (in distilled | Micron-sized Fe | 1.26-1.33 |
| water) | Nano-sized Fe | 64.16-67.67 |
| Soil/groundwater | Micron-sized Fe | 1.07-1.12 |
| | Nano-sized Fe | 69.28-72.65 |

Iron Nanoparticles for Dechlorinating Chlorinated Aliphatic Compounds

The environmental chemistry of metallic or zero-valent iron has been extensively studied. One of the best-documented examples is dechlorination and hydrogenation of chlorinated hydrocarbons (RCI), as summarized in equation (12):

RCl+Fe^{o}H⁺ [symbol] RH+Fe²⁺+Cl⁻ (12)

Research at Lehigh University has examined a large number of chlorinated compounds (Table 9). Most of them can be quickly dechlorinated by the iron nanoparticles.

According to an exemplary embodiment, the invention provides a method for dehalogenating one or more halogen containing compounds comprising the steps of combining the biosolids composition with iron particles having diameters between 1 to 200 nm, and lowering or eliminating the halogen content, as compared to an untreated biosolids composition. Treatment of hexachlorocyclohexanes (“HCHs”), one of the most widely used pesticides, serves as an example of the application of iron nanoparticles for treatment of persistent organic contaminants. Chlorinated pesticides have been widely used as insecticides, fungicides, and herbicides. These pesticides have been discovered to have harmful side effects as they do not readily degrade in nature and tend to accumulate in fatty tissues of most mammals. Perhaps the most infamous of all chlorinated pesticides is DDT. Other compounds may include hexachlorobenzene, chlordane, and dieldrin. These compounds are all amenable for degradation by iron nanoparticles.

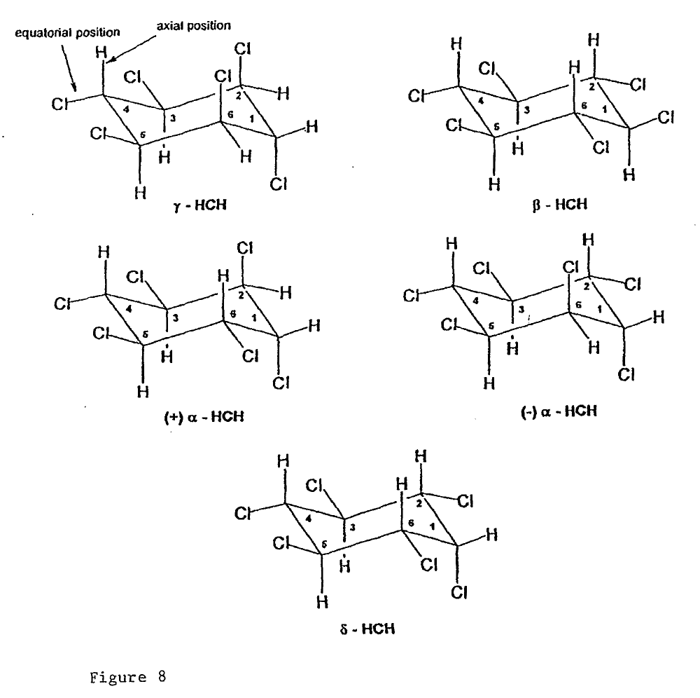

HCHs are a well-known and widely studied class of organochlorine pesticides. FIG. 8 summarizes structures of environmentally significant HCH isomers, including the two HCH enantiomers. Four isomers, namely gamma, alpha, beta, and delta HCHs (FIG. 8), are of major environmental concern due to their toxicity, sorption and bioconcentration potential, and relative stability within the environment.

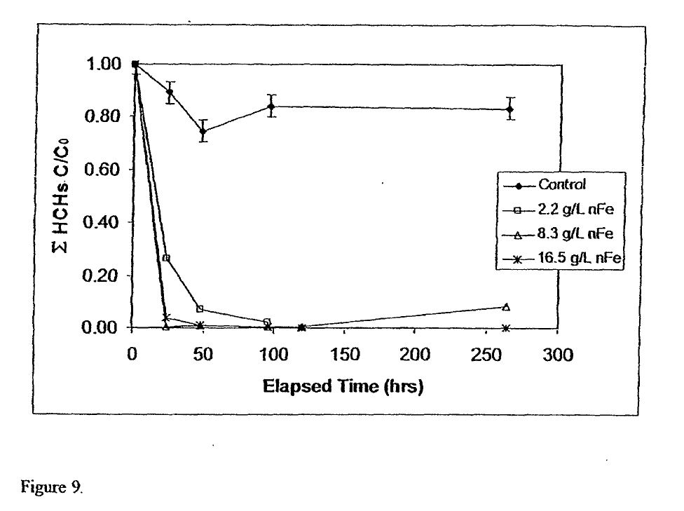

In a laboratory study, groundwater and aquifer samples from a site contaminated by HCHs were exposed to the nanoscale iron particles in batch reactors. The total HCH burden in site groundwater was approximately 700 μg/L. FIG. 9 summarizes the effectiveness of annoscale iron particles in removing HCHs from solution as a function of dosage. In general, batch experiments with 2.2-27.0 g/L iron nanoparticles showed that more than 95% of the HCHs were removed from solution within 48 hours (FIG. 9). The observed pseudo first-order rate constants (k_{obs}) were in the range of 0.04-0.65 hr⁻¹.

TABLE 9
Common environmental contaminants that can be degraded
by the nanoscale iron particles.

| | |
|---|---|
| Chlorinated Methanes | Trihalomethanes |
| Carbon tetrachloride (CCl₄) | Bromoform (CHBr₃) |
| Chloroform (CHCl₃) | Dibromochloromethane (CHBr₂Cl) |
| Dichloromethane (CH₂Cl₂) | Oichlorobromomethane (CHBrCl₂) |
| Chloromethane CH₃Cl | |
| Chlorinated Benzenes | Chlorinated Ethenes |
| Hexachlorobenzene (C₆CL₆) | Tetrachloroethene (C₂Cl₄) |
| Pentachlorobenzene (C₆HCl₅) | Trichloroethene (C₂HCl₃) |
| Tetrachlorobenzenes (C₆H₂Cl₄) | cis-Dichloroethene (C₂H₂Cl₂) |
| Trichlorobenzenes (C₆H₃Cl₃) | trans-Dichloroethene (C₂H₂Cl₂) |
| Dichlorobenzenes (C₆H₄Cl₂) | 1,1-Dichloroethene (C₂H₂Cl₂) |
| Chlorobenzene (C₆H₅Cl) | Vinyl Chloride (C₂H₃Cl) |
| Pesticides | Other Polychlorinated Hydrocarbons |
| DDT (C₁₄H₉Cl₅) | PCBs |
| Lindane (C₆H₆Cl₆) | Pentachlorophenol (C₆HCl₅O) |
| Organic Dyes | Other Organic Contaminants |
| Orange II (C₁₆H₁₁N₂NaO₄S) | N-nitrosodiumethylamine (NDMA) |
| Chrysoidin (C₁₂H₁₃ClN₄) | (C₄H₁₀N₂O) |
| Tropaeolin (C₁₂H₉N₂NaO₅S) | TNT (C₇H₅N₃O₆) |

Reducing Pathogens Using Metal Nanoparticles

Pathogenic organisms in biosolids may be excreted by human beings and animals who are infected with disease or who are carriers of a particular infectious disease. The pathogenic organisms in biosolids can be classified into four broad categories: bacteria, protozoa, helminthes, and viruses. Examples of pathogenic organisms found in biosolids are listed in Table 3.

Federal regulations for biosolids management (Part 503 pathogen-reduction requirements) are divided into Class A and Class B categories. The goal of Class A requirements is to reduce the pathogens in the biosolids (including Salmonella sp. Bacteria, enteric virus, and viable helminth ova) to below detectable levels. When this goal is achieved, Class A biosolids can be land applied without any pathogen-related restrictions on the site. The goal of the Class B requirements is to ensure that pathogens have been reduced to levels that are unlikely to pose a threat to public health and the environment under specific use conditions. The Part 503 has strict restrictions for the use of Class B biosolids, therefore, effort has been focused on producing relatively clean Class A biosolids.

According to an exemplary embodiment, the invention provides a method for stabilizing and reducing or eliminating one or more pathogens in a biosolids composition comprising the step of: combining a biosolids composition with iron particles having diameters between 1 to 200 nm to form a stabilized biosolids composition, as compared to an untreated biosolids composition. According to a separate embodiment, the invention provides a method for removing or eliminating odors associated with one or more pathogens in a biosolids composition comprising the step of: treating a biosolids composition with iron particles having diameters between 1 to 200 nm, reducing or eliminating amounts of odorous biological and organic compounds, as compared to an untreated biosolids composition. The presence of iron nanoparticles should have both direct and indirect impacts on microorganisms. Direct impacts might include uptake, transformation and accumulation of iron nanoparticles by microorganisms. Direct exposure of large dose of iron nanoparticles can cause detrimental effect on microorganisms as iron nanoparticles can penetrate cell membranes and bind to proteins and DNAs. According to an exemplary embodiment, the oxidizable iron particles react with one or more pathogens in the biosolids composition to minimize, de-activate or control odors associated with said one or more pathogens. According to a separate exemplary embodiment, the oxidizable iron particles reduce and immobilize or eliminate odor-producing hydrogen sulfide in pathogens. According to another embodiment, the oxidizable iron particles increase biosolids pH relative to an initial biosolids pH to inhibit growth of one or more pathogens.

Indirect impact entails the reduction of organisms as a result of rapid changes in environmental conditions (e.g., low Eₕ). The reactions of iron with water and other oxidants in water (e.g., oxygen, nitrate, sulfate etc.) produce a rapid decrease in solution standard potential (Eₕ) and increase in pH. A drastic change in the water chemistry including the depletion of dissolved oxygen, could lead to death or at least slow the growth of many microorganisms, especially aerobic microorganisms which respire on dissolved oxygen in water. According to an exemplary embodiment, a method is included for increasing, controlling or maintaining pH of a biosolids composition comprising the step of combining the biosolids composition with iron particles having diameters between 1 to 200 nm, as compared to an untreated biosolids composition.

The presence of iron and generation of dissolved iron also accelerates the aggregation of soil particles and thus dewatering. This hardens biosolids, reduces the biodegradability of biosolids, and diminishes the attractiveness to potential vectors.

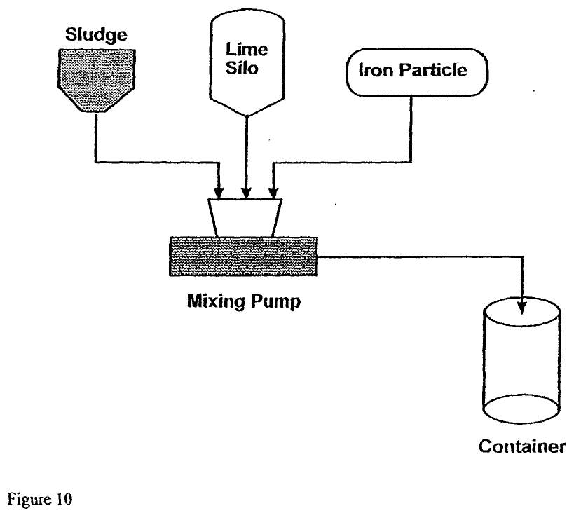

Iron can be added to the biosolids during the sludge treatment (FIG. 10) or after the treatment at the land application location. FIG. 10 is a schematic diagram of an iron particle of the invention feeding and miring system in combination with conventional lime stabilization. The iron can be added as dry powder or dispersed in water as a liquid slurry. The slurry is then mixed with the biosolids.

The amount of iron needed per unit of biosolids mass depends on the solid concentration of the sludge, the amount of sulfur and other elements in the biosolids, and also on the size and activity of the iron nanoparticles. The proper dose can be determined by appropriate laboratory tests.

Iron nanoparticles have several advantages over conventional methods for biosolids stabilization. Iron nanoparticles are multi-functional with respect to treatment of a large variety of pollutants. Iron nanoparticles have been shown to be effective for the transformation of a very large number of organic and inorganic pollutants in groundwater and soil. They are also effective for the reduction of odor-producing compounds, immobilization of heavy metal ions, and extermination of disease-causing pathogens in biosolids.

Iron nanoparticles have high surface reactivity and rapidly react and stabilize biosolid compositions. In a typical wastewater treatment plant, final blending and processing of concentrated biosolids takes from minutes to a few hours. A highly reactive stabilizing reagent is therefore desired. Because of their extremely small size, iron nanoparticles can penetrate the intra-aggregate pores of biosolids and have much better access and availability toward pollutants within the solid phase. The high reactivity can be attributed to: (1) high specific surface, and (2) high reactivity per unit surface area.

Iron nanoparticles have a low adverse environmental impact and are benign to the environment. Iron is the fifth most used element of the periodic table in daily activities; only hydrogen, carbon, oxygen and calcium are typically consumed in greater quantities. Iron is at the active center of many biological molecules and is therefore considered an essential element for life. The end products of iron nanoparticle reactions are iron hydroxides and oxides with no evidence suggesting any negative impact.

Iron nanoparticles are simple to use and are attractive from an economics standpoint. The application processes using nanoparticles are highly portable, and can be combined to various procedures already in place (e.g., heat treatment, biological digestion, lime neutralization etc.). For example, many wastewater treatment facilities have facilities for lime addition and mixing so no new equipment is needed to deploy iron nanoparticles. Little capital investment is therefore required for such conversions.

What is claimed:

1. A stabilized biosolids composition comprising: oxidizable iron particles having diameters between 1 to 200 nm and having specific surface areas from 1000 to 763,358 m²/kg.

2. The stabilized biosolids composition of claim 1 wherein the oxidizable iron particles react with one or more pathogens in the biosolids composition to minimize, de-activate or control odors associated with said one or more pathogens.

3. The stabilized biosolids composition of claim 1, wherein the pathogens are selected from the group consisting of: bacteriums, E. Coli, Legionella pneumophila, Salmonella, protozoa, Cryptosporidium parvum, Giardia Iamblia, Balantidium coli, Helminthes, Ascaris lumbricoides, Enterobius vermicularis, T. solium, viruses, Adenovirus, enteroviruses, Hepatitis A virus and combinations of pathogens.

4. The stabilized biosolids composition of claim 1, wherein the oxidizable iron particles have reduced, and immobilized or eliminated, odor-producing hydrogen sulfide in pathogens.

5. The stabilized biosolids composition procedure of claim 1, wherein the oxidizable iron particles have reduced levels of, or eliminated, odor-producing organic dimethylsulfur compounds.

6. The stabilized biosolids composition of claim 1, wherein the oxidizable iron particles have reduced levels of, or eliminated, chlorophenols.

7. The stabilized biosolids composition of claim 1, wherein the said oxidizable iron particles have reduced levels of, or dechlorinated, chlorine containing compounds selected from the group consisting of polychlorinated biphenyls (PCBs), organochlorine pesticides, 4,4′-(2,2,2-trichloroethane-1,1-diyl)bis(chlorobenzene) (DDT), lindane, chlordane dieldrin, dechlorinating chlorinated methanes, tetrachloromethane (CCl₄), trichloromethane (CHCl₃), dichloromethane (CH₂Cl₂), chloromethane (CH₃Cl), chlorinated ethenes, tetrachloroethene (C₂Cl₄), trichloroethene (C₂HCl₃), trans-dichloroethene (t-C₂H₂Cl₂), cis-dichloroethene (c-C₂H₂Cl₂), chloroethene (C₂H₃Cl), other chlorinated aliphatic compounds, such as I,I,I-trichloroethane (I,I,I-C₂H₃Cl₃), hexachloroethane (C₂Cl₆), chlorinated aromatic compounds, hexachlorobenzene (C₆Cl₆), pentachlorobenzene (C₆HCl₅), tetrachlorobenzene (C₂H₂Cl₄) and combinations of chlorine containing compounds.

8. The stabilized biosolids composition of claim 1, wherein the oxidizable iron particles have reduced, and immobilized or eliminated, toxicity associated with metal ions selected from the group of metals consisting of: cadmium, copper, lead, nickel, cobalt, mercury, chromium, and combinations of metal ions.

9. The stabilized biosolids composition of claim 1, wherein the oxidizable iron particles have increased biosolids pH relative to an initial biosolids pH to inhibit growth of one or more pathogens.

10. The stabilized biosolids composition of claim 9, wherein the oxidizable iron particles have increased biosolids pH from between pH 6 to pH 10.

11. The stabilized biosolids composition of claim 1, wherein the oxidizable iron particles have maintained biosolids pH for time periods greater than two hours.

12. The stabilized biosolids composition of claim 1, wherein the oxidizable iron particles have reduced the oxidation-reduction potential, Eₕ, to a value less than zero.

13. The stabilized biosolids composition of claim 1, wherein the oxidizable iron particles inhibit growth of one or more pathogens.

14. The stabilized biosolids composition of claim 1, wherein the oxidizable iron particles reduce vector attraction potential.

15. The stabilized biosolids composition of claim 1, treated with a conventional procedure selected from the group consisting of alkaline stabilization of biosolids and heat drying stabilization of biosolids.

16. The stabilized biosolids composition of claim 1, wherein the oxidizable iron particles have released water soluble iron cations and other forms of water soluble iron compounds beneficial to plant growth.

17. The stabilized biosolids composition of claim 1, further comprising other metal particles having diameters from 1 to 200 nm.

18. The stabilized biosolids composition of claim 1, further comprising other metal particles having diameters from 200 nm to 100,000 nm.

19. A method for stabilizing and reducing or eliminating one or more pathogens in a biosolids composition comprising the steps of: combining a biosolids composition with iron particles having diameters between 1 to 200 nm to form a stabilized biosolids composition; and inhibiting the growth of the one or more pathogens in the stabilized biosolids composition.

20. A method for removing or eliminating odors associated with one or more pathogens in a biosolids composition comprising the steps of: treating the biosolids composition with iron particles having diameters between 1 to 200 nm; and reducing or eliminating amounts of odorous biological and organic compounds in the treated biosolids composition, as compared to an untreated biosolids composition.

21. A method for dehalogenating one or more halogen containing compounds in a biosolids composition comprising the steps of: treating the biosolids composition with iron particles having diameters between 1 to 200 nm; and lowering or eliminating the halogen content of the treated biosolids composition, as compared to an untreated biosolids composition.

22. A method for increasing, controlling or maintaining pH of a biosolids composition comprising the steps of: treating the biosolids composition with iron particles having diameters between 1 to 200 nm; and monitoring pH of the treated biosolids composition, as compared to an untreated biosolids composition.
